(19) United States
(12) Patent Application Publication
(10) Pub. No.: US 2021/0249122 A1
(43) Pub. Date: Aug. 12, 2021

(54) VALUE-BASED SCHEDULING SYSTEM

(71) Applicant: Steven L. Sholem, Phoenix, AZ (US)

(72) Inventor: Steven L. Sholem, Phoenix, AZ (US)

(21) Appl. No.: 17/233,892

(22) Filed: Apr. 19, 2021

Related U.S. Application Data
(63) Continuation of application No. 16/141,151, filed on Sep. 25, 2018.
(60) Provisional application No. 62/562,908, filed on Sep. 25, 2017.

(51) Int. Cl. G16H 40/20; G16H 10/60; G06Q 40/02; G16H 50/70; G06Q 40/08; G06N 20/00

(52) U.S. Cl. CPC G16H 40/20; G16H 10/60; G06N 20/00; G16H 50/70; G06Q 40/08; G06Q 40/025

(57) ABSTRACT

Methods and apparatus for tracking and evaluating the relative value of medical services provided to patients associated with third party payors (“TPPs”). Under various embodiments of the present invention, the relative value of medical services is considered in evaluating whether to accept a new patient, when and for how long to schedule a patient appointment, and how long a physician should meet with the patient. Methods and apparatus for improving the efficiency of a medical office are also disclosed whereby a physician may more effectively supply a patient with medical services and collect payment for services provided.

### CROSS REFERENCE TO RELATED APPLICATION[S]

This application is a continuation of earlier filed U.S. patent application entitled “VALUE-BASED SCHEDULING SYSTEM,” Ser. No. 16/141,151, filed Sep. 25, 2018, which claims priority to U.S. Provisional Patent Application entitled “VALUE-BASED SCHEDULING SYSTEM,” Ser. No. 62/562,908, filed Sep. 25, 2017, the disclosures of which are hereby incorporated entirely herein by reference.

### BACKGROUND OF THE INVENTION

### Technical Field

The present invention relates generally to determining and tracking a relative value of services provided by medical practitioners and staff. More particularly, the present invention relates to coordinating and access to medical services based on net present value or grade of a patient, other patient related data, and considering the insurance payment behavior.

### State of the Art

The conventional practice of medicine is the result of centuries of improvements in medical technology. Yet, even as medical technologies have improved, the foundation of the medical practice has remained the same. Patients schedule an appointment, are greeted when they arrive for their appointment, and then meet with one or more physicians or other medical services providers. Medical services providers evaluate each patient, diagnose any problems, and recommend tests, prescriptions and other medical procedures as necessary. The patient is then charged for the medical services provided.

Another aspect of medical practice relates to medical services providers' relationships with insurance companies, medical managed care organizations or other third party payors (“TPPs”). As used herein, the term “TPP” is intended to include any organization through which one or more patients receive medical services to be billed through a common payment manager which may pay all or a portion of the charges to a medical practice or facility. Examples of TPPs include, but are not limited to, insurance companies, health maintenance organizations (“HMOs”), physician-hospital organizations (“PHOs”), managed services organizations (“MSOs”), preferred provider organization (“PPOs”), accountable care organizations (“ACOs”), various physician alliances, physician-hospital and physician-medical facility agreements, and Medicare, Medicaid or other indigent, uninsured or under-insured payor supplement organizations.

A TPP may enter into separate or joint agreements with physicians and other medical services providers. Then, through agreements with patients in exchange for risk-adjusted paid premiums paid to the TPP, the TPP pays all or part of a patient's medical expenses. The level of care (i.e. type of service, access to service, duration of service, type and amount of medication, etc.) is adjusted by the TPP to set premiums and determine profitability. Medical services providers, based on their agreements with a particular TPP, agree to charge no more than a specified rate for each type of medical service provided according to a predetermined fee schedule. In exchange for agreeing to the predetermined fee schedule, medical services providers are placed on the TPP's list of preferred providers, or some other list which may determine what portion of the allowable fees the TPP will pay and what, if any, portion of the allowable fees the patient will pay. Other TPPs may agree to pay for all or a portion of medical services regardless of which medical services provider the patient visits. As used herein, the term “medical services provider” is intended to include one or more medical practitioners of any medical field or specialty which may have an opportunity to bill for medical services provided through a TPP. The term “medical services provider” specifically includes, but is not limited to, physicians in any medical field or specialty, nurses, medical assistants and other medical staff such as medical administration and counseling, and any offices, groups or groups of associated offices employing one or more physicians, independent medical contractors, nursing facilities, long and short-term care facilities, off-site providers (home care providers), occupational and physical therapists, behavioral health providers and ambulatory care facilities.

Some TPPs are formed as for profit businesses and generate profits from coordinating the delivery of medical care and there are benefits to medical service providers who sign agreements with TPPs. Particularly in more recent years, however, medical services providers who charge for services through TPPs have experienced problems.

Attempts have been made to increase medical provider income by streamlining medical practices through medical management systems. FIG. 1 illustrates a flow diagram of a conventional medical services process such as that employed by a medical services provider dealing with a TPP. For many TPP plans, prior to visiting a specialist, a referral from a primary care practitioner is required. The primary care physician must request permission for a referral from the TPP. The TPP must then issue a formal approval for a referral to the requesting physician/service provider. The authorization must also be in the specialists' office prior to a patient's visit to the specialist. Many authorizations state that the TPP's approval does not guarantee payment. Without the formal approval, however, no payment will be made to the specialist for medical services provided. More than 98% of referral requests are eventually approved, but the wait to obtain an approval may extend several weeks. The result of such approval requirements may significantly delay the delivery of health care, potentially harm the patient, and delay compensation for the medical services provided.

With a proper referral 2, if required, authorization 4 from the TPP for the medical services requested must be obtained. Conventionally, authorization 4 is accomplished by a medical services provider staff member contacting the TPP by phone, Internet or facsimile to exchange information regarding a patient requesting medical services. The exchanged information typically includes such information as the TPP plan with which the patient is associated, the type of services requested, and the name of the medical services provider who will provide the services. The TPP may refuse authorization or automatically authorize specified services, such as routine physician visits, based on the contract terms.

Once authorization 4 is granted, or in conjunction therewith, a patient's demographics 6 are recorded in the patient's records. To record a patient's demographics, conventionally, a patient completes a form including such information as the patient's name, addresses, relevant numbers, guarantor, employer or TPP information, summary of medical history, allergies, and the like. Once all or part of a patient's demographics are recorded 6, or in conjunction therewith, the patient is scheduled 8 for an appointment. The decision of when to schedule a patient for an appointment conventionally involves such factors as: the type of services requested, medical services provider availability, medical office resources availability and patient condition urgency. After an appointment is scheduled 8, the patient's relevant medical records are retrieved 10 prior to the patient's appointment.

At the time of the patient's appointment, the patient is welcomed by medical office staff and signs-in 12. Sign-in 12 signals to the medical staff that the patient has arrived, and typically also involves collecting a co-pay amount from the patient. The exact amount of the co-pay, whatever it may be, must be determined and collected prior to providing medical services. Co-pay amounts vary considerably and can fluctuate without warning. Sign-in 12, however, may also involve a more detailed record by the patient of the patient's medical history, a description of symptoms, or other patient demographics as needed. Various medical services providers request and retrieve different information from patients at different times throughout the process of providing medical services. When a patient's turn to be seen has arrived, the patient is conventionally greeted by a nurse or medical assistant who confirms basic patient information such as name, address, insurer and purpose of visit, and prepares the patient to be seen by the primary medical services provider, such as a physician or a nurse practitioner, for example by checking the patient's weight, blood pressure, pulse, medications, etc.

The patient is then seen by a primary medical services provider 16, such as a physician, who evaluates the present complaints of the patient or otherwise responds to the purpose for the patient visit, such as by performing a routine physical, the primary medical services provider diagnoses any problems found during the examination, recommends any treatment for problems found, prescribes any medications, procedures, tests, surgery, or the like, and explains the patient's condition to the patient. Either simultaneously with or subsequent to meeting with the patient, the primary medical services provider either dictates for later transcription, or otherwise records a report to the file describing the examination, diagnosis, recommendations for treatment, prescriptions and the like. A copy of the report is generated, signed and sent to the referring entity as well as being filed in the patient's records.

Following the patient's visit, the medical services provider bills 18 the patient, either directly or through the patient's TPP. Completed medical services are typically “checked-off” on a printed form and sent to a data entry clerk to enter diagnoses, codes and “list” prices into the existing office accounting system. Charges are forwarded to the TPP at the billing clerk's convenience. Once the TPP receives the charges, they are reviewed and eventually paid according to the rules and policies of the TPP who may pay according to their fee schedules on a time frame based on their cash flow requirements. Each office independently verifies payment accuracy and follows-up on late payments or non-payments. Gross charges are posted to a traditional accounts receivable system. Payments, discounts and write-offs are entered as received in the “explanation of benefits.” The operation of medical services providers, including the details of the process as illustrated in FIG. 1, is well known to those of ordinary skill in the art.

Conventional medical management systems presently sold focus on accepting patient demographics, scheduling patient visits, and creating charges and submitting them to a TPP or other payor. While a number of systems are available, most concentrate on a traditional accounts receivable system. These systems do not attempt to track payments, nor do they assist in more efficient time management based on a value of the medical services provided against the resources required to deliver those services.

Fee schedules may be provided by an insurer. Such fee schedules are independently produced by TPPs and may or may not be linked to “official” Medicare or other fee schedules. More importantly, however, the allowable fee schedule amounts have very little, if anything, to do with the actual value of the promise of future payment by a particular TPP to a medical services provider. Because each TPP has a different method, timing, and strategy for payment, has a different financial strength behind the promise of payment, and has a different risk of becoming insolvent before providing payment, each TPP's promise for payment does not actually have the same present value.

Additionally, conventional medical management systems still include many activities which may be improved upon to enable medical practitioners to more efficiently and effectively treat patients. Therefore, it is desirable to have a medical management system which intelligently schedules patient visits and evaluates the efficiency of a medical practice based on a more reliable measurement of the value of the patient's payment habits. It is further desirable to simplify medical practice activities to increase efficiency and decrease fraud losses and, therefore, increase profits for medical practitioners.

### SUMMARY OF THE INVENTION

The present invention provides a medical management system which considers a relative value of services provided to patients by a medical services provider. Elements of the system taught in U.S. Pat. No. 7,702,522 may be utilized within the medical management system disclosed in this application and therefore U.S. Pat. No. 7,702,522 is incorporated entirely herein by reference.

As used herein, the term “net present value” is intended to include any estimated or actual value calculated as a function of an actual or estimated cost of collecting the value such as a time cost, resources cost, inflation cost, risk allowance cost or any other cost and/or a desired profit margin. In particular embodiments of the invention, the relative value of services is an estimated net present value (“NPV”) of services for patients associated with a particular third party payor (“TPP”). The relative value of the services provided is evaluated when determining whether to accept a new patient, whether to enter into a medical services agreement with a TPP, whether to schedule an appointment and for how long the appointment should last, which resources to reserve for the appointment, and how long a particular medical services provider should spend with a patient at the time of the appointment based at least partially on the type of procedure to be performed or the type of medical services to be rendered. The NPV of services is essentially the value of the services calculated as if payment were received today. The NPV of services considered takes into account the payment patterns of a TPP including, but not limited to, how long from the time of service it takes to collect payment from the TPP, what the allowable charges of the TPP are, and what percentage of the allowable charges for a particular service the TPP typically pays. The NPV of services considered may also account for lost investment opportunities, inflation, and administrative costs in tracking and collecting the future payments. Other relative value amounts may include additional information in conjunction with the NPV for appropriate determinations. Other relative value amounts may be calculated as a function of the operating costs and administrative costs of a particular medical services provider, the break-even point for particular services, a desired profit margin, and the apparent stability of a TPP based on trends in the TPP's payment patterns.

In response to a request for a medical services provider to enter into an agreement with a TPP, accept a new patient, schedule an appointment, or visit with a patient, an indicator is generated to express the desirability of the action or otherwise indicate an estimated profitability or relative value of the requested action to the particular medical services provider. The indicator may be determined by analyzing, but not limited to such things as: 1) whether the patient is a cash patient; 2) whether a credit card is on file; 3) the available credit of the credit card on file; 4) patient credit score; 5) TPP's NPV; TPP's weighting; 6) Health Savings Account/Medical Savings Account (HSA/MSA) balance; 7) patient amount due; 8) prior balance due; and 9) pre-payment amount With the appropriate indicator available, a medical services provider may more appropriately and effectively make decisions on future actions which have an effect on the profitability of the medical services provider's business. It is also contemplated that the parameters of a particular agreement, appointment, or other action, such as the duration of an appointment, may be adjusted prior to the medical services provider agreeing to the action, to increase the likelihood that the action will be profitable for the medical services provider and to maximize the profitability of dealings with a particular TPP. In one specific embodiment, a primary medical personnel, such as a physician, is provided with a timer during a visit with a patient to indicate a recommended visit duration within which the physician may still “break-even”, or more preferably make a profit, on the visit.

Corresponding software, hardware and interrelated systems enable the various embodiments and aspects of the present invention by storing TPP and statistically significant sampling of payment pattern histories and related data from a plurality of medical services providers in a common location to increase the usefulness of the information. According to embodiments of the present invention, at any time, a medical services provider may access appropriately configured software to generate a report on the real-time profitability of the medical services provider's business generally, or specifically, the profitability of relations with a particular TPP. The medical services provider may also generate graphs or other reports illustrating outstanding payments due by individual TPPs, how long the payments are overdue, and when payments are expected based on the payment patterns of the TPPs. It is further contemplated that by tracking the payment patterns of a TPP over time, and analyzing the payment pattern trends of a TPP, it may be predicted when a TPP is struggling financially and likely to become insolvent. The unique predictive ability of this system allows an early warning to medical services providers which reduces a TPP's ability to hide pending insolvency and allows medical services providers to better evaluate whether the TPP is attempting to receive medical services for their patients without the intention of properly compensating the medical services providers. In specific embodiments of the present invention, appropriately configured and accessible databases are available through the Internet to enable access to relevant data from any appropriately configured computing device, such as, but not limited to, a tablet, a smartphone, a mobile computing device, a desktop, a laptop or other personal computer having software for accessing the appropriate databases and performing the required calculations.

The foregoing and other features and advantages of the present invention will be apparent from the following more detailed description of the particular embodiments of the invention, as illustrated in the accompanying drawings.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 includes a flow diagram of a prior art medical services process;

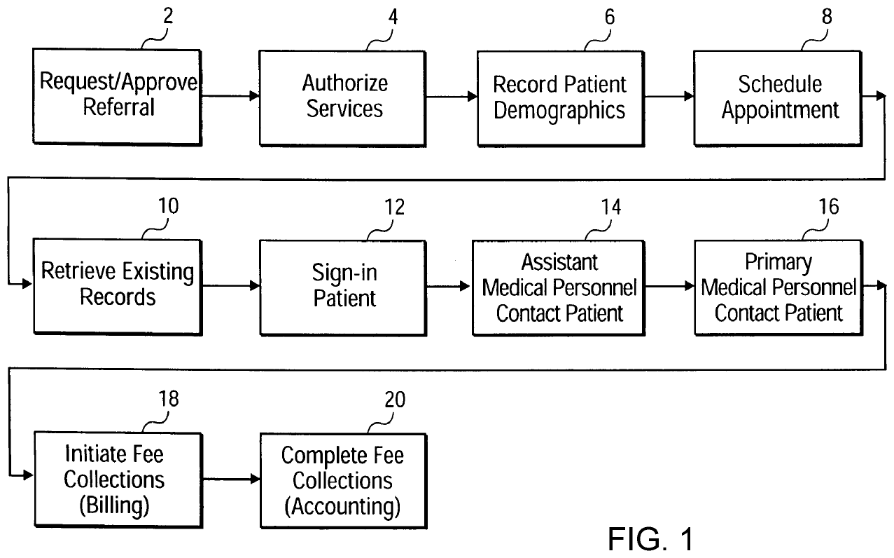

FIG. 2 includes a graph of the present value of medical services provided as a function of time until payment is collected;

FIG. 3 includes a block diagram of the flow of money from patients to medical services providers;

FIG. 4 includes a general system diagram illustrating a medical management system according to an embodiment of the present invention;

FIG. 5 includes a flow diagram illustrating a medical management process for each contacted patient according to an embodiment of the present invention;

FIG. 6 includes a flow diagram illustrating a process for determining whether to accept a new patient;

FIG. 7 includes a flow diagram illustrating a process for generating a risk indicator;

FIG. 8 includes a flow diagram illustrating a process for determining when to schedule an appointment; and

FIG. 9 includes a flow diagram illustrating a process for submitting physician superbills data for payment.

### DETAILED DESCRIPTION OF EMBODIMENTS OF THE INVENTION

The relative value of providing medical services to a patient may vary within a wide range of values for each of a variety of medical services providers. Additionally, as a function of the payment patterns of a variety of third party payors (“TPPs”), the relative value of providing the same medical service to a variety of patients having different TPPs may vary within a wide range of values. One example of a relative value calculation is the net present value (“NPV”) of services. The concept of NPV relies, in part, on the principle that whenever services are performed in exchange for a promise of future payment, those providing the services are, in essence, granting a loan to those receiving the services until payment is made. In systems where billing for services is done periodically, such as for medical services, that loan is traditionally interest free if paid within a predetermined period. However, the value of a payment received at some point in the future is less than the value of the same payment received now. This decrease in value over time is due to numerous factors including, but not limited to, inflation, lost interest bearing investment opportunities (the time value of money), risk of default or risk of non-payment, and administrative costs in tracking and collecting the future payment. Such calculations are well known in the art and may readily be performed by economists, accountants or financial analysts of ordinary skill in the art, using well-known equations. Further, in addition to the NPV of a TPP, other factors must be considered in order to determine the relative value associated with providing medical services to a patient.

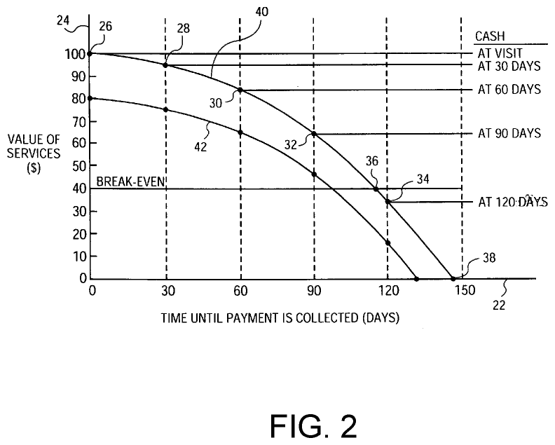

FIG. 2 includes a graph illustrating how payment received at various times in the future may vary the value of the payment to a medical services provider at the time the services are provided. For the graph shown in FIG. 2, the horizontal axis 22 represents time which will pass until payment is collected for services provided today, and the vertical axis 24 represents the value of the services provided today calculated as if paid today. Points 26, 28, 30, 32, 34, 36 and 38 along the arcing line 40 represent the actual NPV of a medical service performed today as a function of when the payment for today's services will actually be collected. As illustrated by the first point 26, if medical services were provided today having a value of $100 and $100 cash was collected as payment at the time of service, the NPV of the $100 is $100. The $100 may be used immediately by the medical services provider for any purpose. If, however, payment is not collected for 30 days, the second point 28 on the arcing line 40, the NPV of the $100 services provided may only be $95. At the time the services are provided, the medical services provider is owed $100 for the resources and time expended to provide the services. Instead of immediately receiving $100, however, the medical services provider must wait 30 days. During those 30 days, inflation has decreased the buying power of the $100 dollars, the medical services provider has missed opportunities to invest the $100 in an interest bearing investment or has paid interest on outstanding debts which could have been paid by the $100, and the medical services provider has been required to expend resources to collect the $100 such as sending out a bill and tracking the status of the payment. In other words, under the present example, the $100 payment received in 30 days has the same value as a $95 payment received today.

As can be seen through the example provided in FIG. 2, the longer the delay before payment is collected, the lower the NPV of the payment to the medical services provider. Intuitively, this decrease is due to greater deflation of the value of money during the longer time, greater lost opportunities for alternative uses for the money, and greater administrative costs in collecting the money as time passes. For example, for a specific medical practice, a $100 payment received at 60 days, the third point 30, may have an NPV of $85, a payment received at 90 days, the fourth point 32, may have an NPV of $65, and a payment received at 120 days, the sixth point 34 on the arcing line 40, may have an NPV of $35. As also shown in the graph of FIG. 2, at some point 38, the NPV of the services will be $0. In other words, payment which will not be received until the sixth point 38 has no present value, and the medical services provider is essentially providing the services for free or at a loss. As should be clear to one of ordinary skill in the art, though readily determinable using well known equations, the actual path of the arcing line 40 for any specific time and medical service provider will vary because the specific characteristics of each service provider are different, and the relevant factors in a relative value calculation may vary over time.

Regardless of when payment is received, however, a medical services provider also incurs expenses by providing the medical services which must be paid. For example, the medical staff who performed the services and the other general office staff required to run a medical services provider office all need salaries and benefits, equipment used in providing the services must be purchased or otherwise paid for, cleaned and/or disposed of, the building in which the services were provided must be paid for, cleaned and updated, electricity and other utilities are needed, medical malpractice and other insurance must be paid, a profit margin is desirable, and many other expenses are required to make the medical services available. Thus, the medical services provider likely cannot afford to maintain the medical services for any significant length of time if the average NPV of payments is below the operating costs of the medical services provider's office. In other words, there is a break-even point 36 long before the NPV reaches $0, beyond which it is unprofitable for a medical services provider to provide services. Additionally, recent experience by medical services providers dealing with TPPs indicates that payment by some TPPs may be less than a full amount, and may require additional administrative expense to collect, thus, further affecting the relative value of the services to the medical services provider. The second arcing line 42 of the graph of FIG. 2 shows an instance where only 80% of the total bill is ever collected.

Because the various TPPs pay their bills differently, and on different payment schedules, some quickly paying their bills in full and others paying their bills late, only partially paying or not paying, it is more profitable for a medical services provider to provide services to those patients who are associated with TPPs which quickly pay their bills in full. By example, consider two medical services providers, each having a break even point of $40 on the arcing curve 40 of the graph of FIG. 2 and providing the same medical services for an average of 100 patients per week. The first medical services provider provides service only to patients of a first TPP which pays billed charges in-full at 30 days from service ($95/$100). The second medical services provider provides service only to patients of a second TPP, which uses the same allowable fee schedule as the first TPP, but which pays an average of 80% of the billed charges at 60 days from service ($66/$100). It should be clear from the graph shown in FIG. 2 that although both the first and second medical services providers are making a profit in their businesses and working for patients of TPPs with identical allowable fee schedules, the first medical services provider is making approximately $29 more profit today, on average, for each $100 in services billed when the NPV of the services is considered.

As illustrated by the previous example, the allowable fee schedule amounts of a TPP have very little to do with the present value of the promise of future payment by the TPP. Nevertheless, in conventional medical services management systems, the allowable fee schedule amounts of a TPP are a primary standard by which decisions to perform medical services are made. Conventional medical services management systems do not consider the relative value of medical services, the NPV or the relations between the relative value of medical services and the cost of operating a medical services provider's office in the determination of whether to accept a patient, how that patient should be scheduled or how much time that patient should be allotted for a visit.

An embodiment of the value-based scheduling system provides a solution to the limitations of the conventional medical services management systems. The value-based scheduling system is a methodology that practices can adapt to qualify a patient's ability to pay for the services they are requesting and further, recommending alternative for those who do not have an ability to pay for services. The value-based scheduling system is designed to keep physicians in practice by decreasing overhead and increasing profitability. It delays or prevents physicians from quitting and allows continued delivery of healthcare by scheduling services based on value as opposed to first-come-first-serve principles.

However, it is no longer enough to determine a relative value of medical services based solely on the NPV. The following Table 1 provides an example, and not a limitation, of factors to consider and how to utilize the information to determine the relative value.

TABLE 1
Factor
Cash Patient
Credit Card on File
Available Credit
Patient Credit Score
Insurance Company NPV
Insurance Company Weighting
HSA/MSA Current Balance
Patient Amount Due
Prior Balance Due
Pre-Payment Amount
Other Relevant Risk Factors

According to a first aspect of the present invention, the relative value of potential medical services to be provided for a patient is calculated based on a plurality of factors in a determination of whether to accept a new patient. In one embodiment of this first aspect, when considering whether to accept a new patient, the following data is processed to determine the relative value: 1) whether the patient is a cash patient; 2) whether a credit card is on file; 3) the available credit of the credit card on file; 4) patient credit score; 5) TPP's NPV; TPP's weighting; 6) Health Savings Account/Medical Savings Account (HAS/MSA) balance; 7) patient amount due; 8) prior balance due; 9) pre-payment amount; and 10) other factors contributing to credit risk. This data is analyzed and evaluated, and the patient is assigned a rank, grade or other indicator to indicate to those considering whether to accept the new patient an estimated relative value of the likely services for the patient. It is contemplated that the rank assigned may be any rank form or style such as a color (e.g. red, yellow and green), a number, scaled number grade or letter (e.g. 1 to 100 or A to F), a graded series of words (e.g. good, better and best), or more simply a brief indicator of acceptance or rejection (e.g. yes and no, or accept and reject). Although there are numerous factors which may be considered in generating a relative value of the services, such as those considered above, other factors to consider may vary for each application, and relevant data may include, but are not limited to, one or more of: the average time for payment in general and for specific services; the average percentage of allowable billed charges paid, in general and/or for specific services; the allowable fees schedule; the number of patients associated with the TPP generally and within a particular region; the required copay amount for this or other patients; the total makeup of patient demographics for the specific services provider; activity-based costs involved in providing the medical services for a patient of the specific TPP; and the like. Preferably, data which is evaluated is regularly updated as additional charges are billed to and paid by TPPs. Most preferably, the data is maintained and updated in substantially real-time by an appropriate processor including software as described later herein. Based at least in part upon the rank assigned the patient's TPP, a decision-maker makes the decision of whether to accept the new patient. Of course, the decision-maker may be a medical services provider, staff member, or may alternatively be an automated decision-maker such as a computing device running appropriate scheduling software having an over-ride option for special circumstances. The system operates software providing a rule to set how many patients of each ranking per day/week/month that may be scheduled for a medical practice, how many new patients to accept per day/week/month by the medical practice, and the like.

In a second embodiment of the first aspect of the present invention, when considering whether to enter into an agreement with a new TPP, or to renew an agreement with a TPP, data relevant to the TPP and a relative value of the services provided to patients of the TPP, such as a NPV, is reviewed and evaluated and the TPP is assigned a rank, grade or other indicator, like with the first embodiment. Also similar to the first embodiment, the rank is based upon data relevant to the TPP and the decision to enter into the agreement is based upon at least a portion of the relevant data and/or the rank.

In other embodiments of the first aspect of the invention, in considering whether to enter into an agreement with a TPP or to accept a new patient, in addition to the data relevant to the TPP used in the first and second embodiments additional information is reviewed and evaluated in determining a relative value and/or a rank for consideration. As with the first and second embodiments of this first aspect, there are numerous other factors which may be considered, not all of which may be listed here. However, some significant data factors may include: data relevant to operation of the particular service provider considering the rank such as overall operating costs and overhead, specific costs for providing specific services, specific services offered by the service provider, accounts payable amounts, accounts receivable amounts, a desired profit margin and the like; and data more generally relevant to society such as an estimated or prevailing inflation rate, an opportunity cost, and the like.

According to a second aspect of the present invention, the relative value of medical services to be provided for a patient is calculated and considered in a determination of scheduling the patient for an appointment. In a first embodiment of the second aspect of the present invention, a rank for a patient, similar to the rank disclosed in the first aspect of the invention, is generated when a patient attempts to schedule an appointment. For example, and with reference to Table 1 above, a cash patient that prepays for the services may receive a rank of 100 and receive medical care on an expedited basis. If the cash patient agrees to pay at the time of service, the rank may be 70 and the patient receives medical care on a preferred basis but not necessarily as quickly as a prepay cash patient. If the patient is not a cash payment, then the system operates to collect various types of data in order to determine the rank of the patient. Table 1 discloses an example of a plurality of factors that may be utilized to determine a patient's rank. For example and without limitation, a non-cash patient analyzed according to the factors provided in Table 1 may include the system processing an algorithm that determines a grade for the non-cash patient by determining subgrades for each factor including at least one or more of the following: 1) Cash Patient Grade; 2) Credit Card on File Grade; 3) Available Credit Grade; 4) Patient Credit Score Grade; 5) TPP's NPV Grade; TPP's Weighting Grade; 6) Health Savings Account/Medical Savings Account (HAS/MSA) Balance Grade; 7) Patient Amount Due Grade; 8) Prior Balance Due Grade; 9) Pre-payment Amount Grade; and 10) Other Factors Contributing to Credit Risk Grade for a Total Scaled Grade or Rank or Relative Value on a scale of 0-100. Other scales may be utilized, such as A-F or any other grading scale. It will further be understood that the algorithms utilized to determine the grading may be controlled by fine tuning weighting or grading per analytics such as standard machine learning which may be calculated at various strata based on the data.

The rank is used by embodiments of the system by a medical services provider in determining if and when the patient will be scheduled for an appointment. Although there is a variety of data which may be used in generating the rank, not all of which may be practically listed here, the data may include such information as: the estimated NPV of the service requested by the patient for the TPP with which the patient associates; the estimated cost of providing that service; the operation costs of the specific service provider; a desired profit margin; the types of services being provided to other patients near similar appointment times; the urgency of the medical condition; the history of the patient with the services provider; and the like. However, retrospective analysis of the TPP's response to an appropriate payment for the patients seen on an emergency basis may form a decision basis for subsequent participation with that TPP. It is also contemplated that data relating to the specific periodic payment dates of a TPP may be considered in a determination of when to schedule a patient such that the patient may be scheduled most optimally near the closing date for the nearest payment cycle.

In a second embodiment of the second aspect of the invention, a relative value of medical services to be provided for a patient is used to determine the scheduling of resources for a patient appointment. When an appointment is scheduled, associated resources such as office equipment, physicians, rooms, and support staff, are also scheduled to enable the medical services provider to competently provide the required services. In this third embodiment, the relative value of the medical service to be provided is considered in scheduling resources and the resources are each assigned a quality or desirability level such that the newest resources, most experienced physicians, largest rooms, etc. are scheduled for those patients associated with TPPs with high rankings, or for those medical services providing the greatest relative value. Although every medical services provider certainly desires to provide the best service and resources to every patient, there are differences in resources even within an office. It may be advantageous to grant use of the best resources by those associated with TPPs who provide the greatest relative value for the medical services provider.

According to a third aspect of the present invention, a NPV of the medical services to be provided is considered by a physician, or other medical services provider employee, in determining the resources the physician should utilize with a patient during an appointment. By specifically indicating the relative value, such as the NPV, of a particular medical service to the physician prior to the physician administering that service, the physician may better evaluate the length of time the physician should spend with that patient. Furthermore, if the physician knows the specific estimated time the physician should spend with the patient to make the visit profitable for the medical services provider, the physician may more efficiently visit with the patient to make the visit profitable. Certainly, however, the indicated time would only be a recommendation and the physician could adjust the actual time spent with a patient as required for a particular patient. In a particular embodiment, the physician, or other medical services provider employee, is provided with a time frame indicating the time remaining on the recommended visit time for a visit with a particular patient.

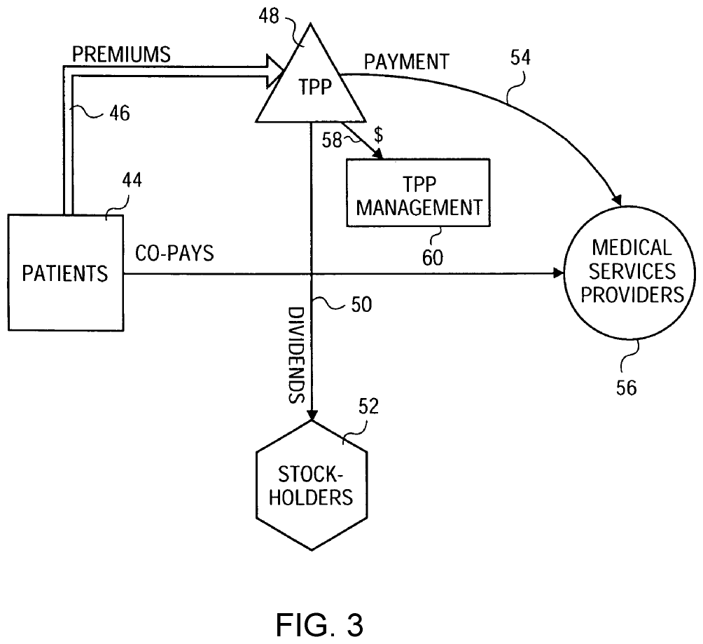

According to a fourth aspect of the present invention, data relevant to a calculation of the NPV for medical services provided to patients of a particular TPP is used to predict the future insolvency of that TPP. Some TPPs are conventionally operated as a business for profit. As a business for profit, a primary concern of TPPs is the profitability of the business. Thus, when profit margins drop, TPPs find ways to bring those profits back up. As illustrated in the drawing of FIG. 3, patients 44 desiring medical services, or insurance for medical services payments, may pay premiums 46 to a TPP 48 in exchange for at least partial payment of future medical bills. With the money from the premiums, TPPs pay dividends 50 to their stockholders 52, money 58 in the form of salaries and bonuses to their management 60 and other employees, and pay 54 medical services providers 56 for medical services provided for the patients 44. The flow of money may continue smoothly until the TPP's 48 payments 50, 54 and 58 to stockholders 52, TPP management 60 and medical services providers 56, exceeds the TPP's income from patient premiums 46.

Struggling TPPs have been shown to decrease their expenditures by delaying payments 54 to medical services providers, providing only partial payments for billed services, denying additional services, denying payments altogether when expenditures begin to exceed income, down-coding claims, increasing premiums, increasing co-pays, decreasing permissible drugs or allowable prescription sizes, slowing authorizations for services, limiting the number of allowable visits, shifting specialty care to primary care physicians, linking TPP enrollment with physician reimbursement, shifting financial risk to physicians, and forcing physicians to see patients after the TPP stops paying. By altering the patterns of their payments to and treatment of medical services providers, TPPs have evidently been able to temporarily extend the life of the TPP until the delayed payments catch up to them, at which point the TPP becomes insolvent. A large majority of any outstanding payments due medical services providers are lost, however, when a TPP becomes insolvent. During the time from when the TPP begins to alter its payment patterns and the time it becomes insolvent, however, a medical services provider typically does not know that the services the medical services provider is providing for patients of the TPP will not be paid. Embodiments of the system capture a plurality of these factors that adjust the grade of the patient based on the payment patterns of the TPP.

It is contemplated, in a first embodiment of this fourth aspect of the invention, that at least a portion of the data used to calculate the NPV of medical services, such as the delay until payment is made and the percentage of the allowable billed fees paid, is tracked over time to provide an indication of when a particular TPP is coming closer to becoming insolvent. In this first embodiment, by tracking the payment history of a particular TPP in its transactions with one or more medical services providers, the point at which the TPP begins a pattern of delaying payments or paying only partial payments may be detected. By detecting such patterns, medical services providers may better evaluate the desirability of dealing with particular TPPs or accepting or treating patients from certain TPPs because of the TPPs' present inability to pay their bills on time.

In particular embodiments of this fourth aspect of the invention, an indication of worsened payment patterns by a TPP is indicated to medical services providers to assist in such decisions as entering into an agreement with a TPP, accepting a new patient of a TPP, scheduling a patient's appointment, and visiting with a patient. In other particular embodiments of this fourth aspect of the invention, a worsened payment pattern is automatically considered as a factor in ranking a TPP or patient, or determining the best duration for a visit with a patient. It is also contemplated that an improved payment pattern may be useful in some situations for evaluating the desirability of entering into an agreement with a TPP, accepting a new patient of a TPP, scheduling a patient's appointment, or visiting with a patient. It is anticipated that by providing medical services providers with an indication of worsened payment patterns by TPPs as a substantially real-time indicator of the financial viability of the TPPs, the payment patterns of TPPs generally will improve. It is also anticipated that an early warning system will place the TPP on notice that its behavior is being monitored in real-time and that unethical accounting practices will be observed. This should hasten the demise of financially inadequate TPPs.

According to a fifth aspect of the present invention, a database is provided for storing, collecting and updating relevant data for calculating the NPV of services as described in relation to the various embodiments of the present invention. The database preferably includes data for one or more, and preferably all, TPPs such as, by example only: the allowable fee schedules; a payment history for each services type; an insolvency indicator; TPP patient demographics, and the like. A separate or an associated database or fixed selection may also include data relating to: the operating costs of one or more specific medical services providers; collection costs; a desired profit threshold; rank indicator parameters; investment interest amounts; inflation amounts; and the like.

In a first embodiment of the fifth aspect of the invention, a database such as that described herein is provided in a stand-alone computer memory such as a hard drive of a conventional laptop or desktop computer. In a second embodiment of the fifth aspect of the invention, the database is stored in a computer network server, mainframe computer, or cloud based server, and accessible from any one of a plurality of local and/or remote computer terminals such as is described later herein. The local and/or remote computer terminals may access the network server through any communication means known in the art including, but not limited to, direct wiring, telephone wiring, radio wave, cellular or other wireless technology, the Internet, or any other method of accessing a computer network server known in the art. In a third embodiment of the fifth aspect of the invention, the database, stored on a computer network server, updates its contents through communication with a plurality of sources including one or more other medical services providers. In this third embodiment, it is contemplated that the data for the TPP payment histories and other TPP-related information may be retrieved from a plurality of medical services providers each subscribing to a service allowing access to the database. By compiling data from numerous sources, a more accurate estimate of the relative value of a particular service, and other data used in generating rankings, etc., may be obtained.

In a conventional medical services provider's office, when a primary medical provider, such as a physician, completes a visit with a patient, the primary medical provider also generates a “superbill” and delivers it to an employee of the medical services provider such as an accounting or data entry clerk. The “superbill” is conventionally a paper record which includes a list of services provided to the patient for billing to the TPP after the information has been appropriately entered into a standard TPP claim. According to an sixth aspect of the present invention, a primary medical personnel records the “superbill” information into a computing device which may be directly downloaded to a billing database and sent to a TPP immediately. By recording the “superbill” information in a form which may be directly downloaded and immediately billed to the TPP, errors from misreading a physician's handwriting or miscopying the information may more easily be avoided, and payment may be received more quickly from the TPP.

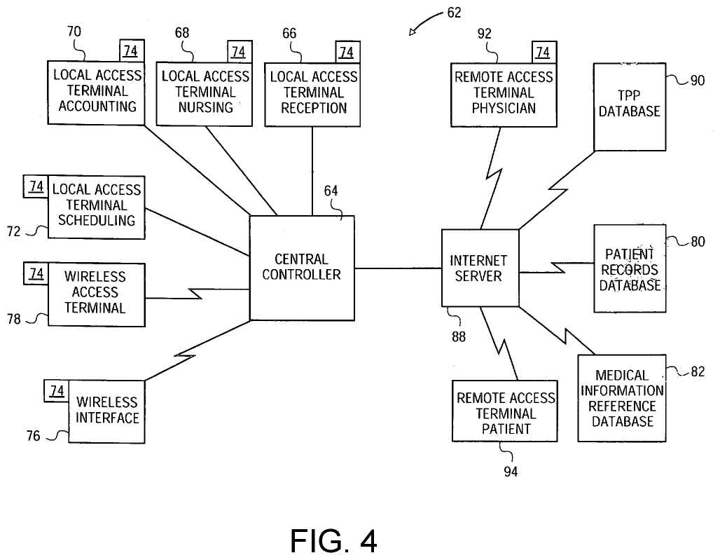

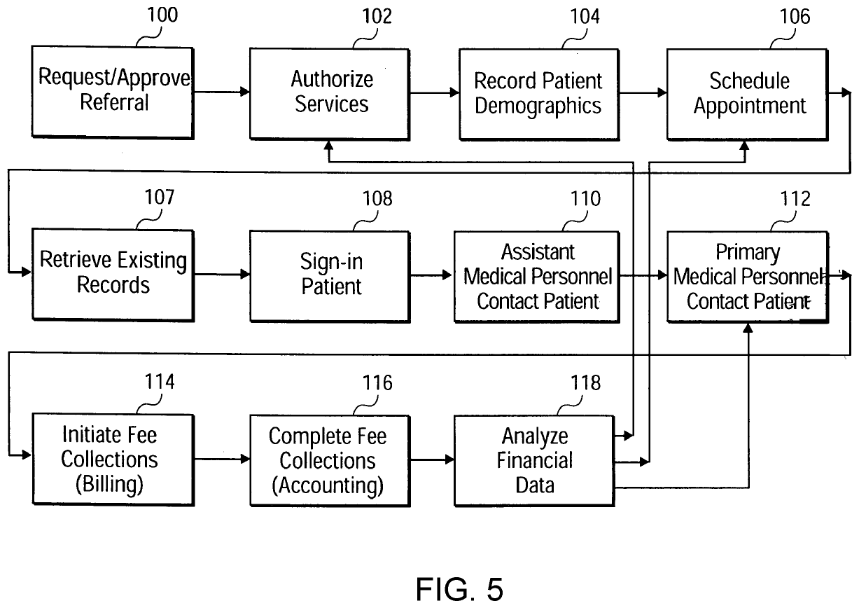

As will be clear to one of ordinary skill in the art, any number of the previously described aspects of the present invention may be incorporated into a system for use by a medical services provider. The following medical management system, as shown and described in FIGS. 4 and 5, is only one example of how the various aspects of the present invention may be implemented in combination.

FIG. 4 shows a block diagram of an embodiment of a medical management system 62 in accordance with various aspects of the present invention. The medical management system includes a central controller 64 for enabling interconnection between the various associated parts of the system 62. The central controller 64 may be configured as a local computer network server, or any other computer network server well known to those of ordinary skill in the art. The operating system supported by the controller will vary depending on the basic operating system selected by a particular medical services provider. Associated with the central controller 64 are a plurality of local access terminals 66, 68, 70 and 72 through which access to the medical management system 62 may be attained. It is contemplated that the hardware for each access terminal is any computing device, both desktop and mobile computing devices. As will be clear to one of ordinary skill in the art, each appropriate access terminal may inherently also include one or more of an associated display device, input device, direct or wireless network connection, printer, or other peripheral device as required to enable the purpose of the access terminal or database. Such peripheral devices are well known to those of ordinary skill in the art.

Software for performing the functions required by each local access terminal 66, 68, 70 and 72 is included on the respective access terminals hard drives. For example, on a local access terminal 72 from which it is desirable to schedule appointments scheduling software configured according to embodiments of the present invention is included, and on a local access terminal 70 from which it is desirable to perform accounting tasks, accounting software configured according to embodiments of the present invention is included.

Also associated with the central controller 64 is one or more wireless interfaces 76 or wireless access terminals 78. The wireless interface includes a microphone and voice recognition capabilities to reduce the need for transcription. Voice recognition software are well known to those of ordinary skill in the art. The wireless interface 76 includes software to enable a physician or other medical personnel to complete forms, update simple documents, record and submit “superbills”, and the like. A computer programmer of ordinary skill in the art will readily be capable of programming the required software given the requirements of a particular system. In more complete embodiments, a wireless access terminal 78 is used by which complete access to the central controller 64 connections may be obtained.

The central controller 64 may also have access to the Internet through an Internet server 88 in communication with the central controller 64. In the present embodiment, the patient records database 80, the medical information reference database 82 and a TPP database 90 are available through an Internet connection so that data which may be needed at more than one location may be more easily accessed by all authorized users. Certainly, it would be in accordance with the various aspects of the present invention if the data from each of the databases 80, 82, and 90 were located at the medical services provider's office, or remotely located at some other location such as a remote server which coordinates access to the databases and provides updated data and other services to its subscribers.

To enable substantially real-time information on the relative value of services provided to a patient of a TPP, and to quickly retrieve patient records, it is preferable only that the data be available for access by an authorized user through appropriately configured software. By having the TPP database 90 and the patient records database 80 available by numerous users at various locations, the information therein may be regularly updated by using data from several locations, making the databases more useful. By having the medical information reference database 82 at a central location and accessible through the Internet, it is not necessary to store the information reference database 82, which is likely to be rather large, at every location.

Through a remote access terminal 92 such as a computer with an Internet connection, a physician may gain access to the central controller 64 for working from a remote location. Patients, too, may access the central controller 64 through a remote access terminal 94 to enable the patient to review the patient's appointment schedule, read medical references, schedule new appointments, and the like. It is anticipated that patients may establish an access account through a medical services provider to gain access to certain data available through the medical services provider's central controller 64.

FIG. 5 is a basic process chart indicating general categories of sub-processes which may occur for each patient contact under embodiments of the present invention. The following example in reference to FIG. 5 is one embodiment of a method referencing many aspects of the process a medical services provider goes through to provide medical services to a patient.

Under an embodiment of the present invention, if a referral 100 is required prior to a medical services provider visiting with a patient, the referring medical services provider contacts the patient's TPP or eligibility data service through the Internet, phone or the like, inputs the appropriate visit type (and associated code number), the patient's name and TPP reference number, and the patient is automatically granted approval, or rejected based on the TPP coverage of the patient's associated TPP plan.

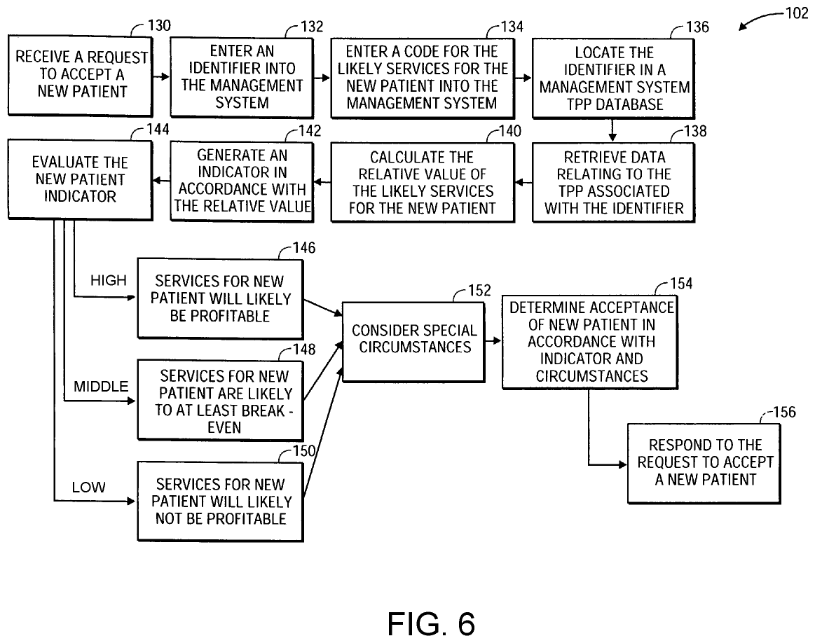

After the TPP has granted the referral request, including the patient's eligibility data on contract payment responsibility, the medical services provider to which the referral was made, or any medical services provider accepting a new patient, must decide whether to accept the new patient and authorize 102 its own medical staff to treat the patient. As shown in the flow diagram of FIG. 6, under an embodiment of the present invention, as part of the authorization process 102, a value-base scheduling system proactively determines whether to accept a patient and when to schedule the accepted patient. This may be accomplished by a medical services provider staff member, such as a receptionist, new patient secretary or scheduling clerk, receives a request to accept a new patient 130 and collects at least a TPP identifier, but preferably more detailed introductory information relevant to the new patient such as the patient's TPP, TPP plan, name, address, gender, age, and the like, and enters the data into a computer terminal in communication with a patient database and evaluation software. The staff member enters the identifying information into the management system 132, and a code for the type of services the patient will likely be receiving 134. For example, if the medical services provider is a gynecology clinic and the new patient is pregnant, a relevant code may be entered. Alternatively, if the medical services provider is a general family practice clinic, and the new patient is a child, a different relevant code may be entered corresponding to the likely services which will be provided to a child as opposed to an adult. Software operating on the staff member's access terminal locates the identifying information in a management system database 136 and associates an appropriate TPP with the identifier to access and retrieve the TPP's data 138. The management system, having evaluation software and using information in a TPP database such as the TPP's previous payment patterns to this and other medical services providers, the estimated NPV and relative value of the likely services to be provided to this patient 140, and the like, generates an indicator of the relative value of the services in accordance with the calculated relative value 142, and provides the medical services provider staff member with an indication of whether it would be profitable for this medical services provider to accept this new patient. The indicator may be the rank determined from the algorithm processing the factor data provided in Table 1. In the example shown in FIG. 6, if the indicator generated is within a predetermined high range, this corresponds to an indication that the services for the new patient will likely be profitable for the medical services provider 146. Contrarily, if the indicator generated is within a predetermined low range, this corresponds to an indication that, based on the considered factors or other reasons, services provided for this new patient will likely not be profitable for the medical services provider 150. If a middle indicator is generated, this may correspond to an indication that services for the patient are at least likely to break-even for the medical services provider 148. The system evaluates the new patient indicator 144 and any other special circumstances 152 which may exist. Special circumstances may include such circumstances as the urgency of the new patient, the identity of the new patient, any additional conditions which may be placed upon this new patient to better ensure profitability for services provided, and the like. The system may then determine whether to accept or deny the new patient 154 and respond to the request 156. If the patient has been seen by other medical services providers also subscribing to the same data tracking service, the patient's information will already be recorded in the system and the data may be confirmed and updated, if needed, and used to obtain an indication of authorization.

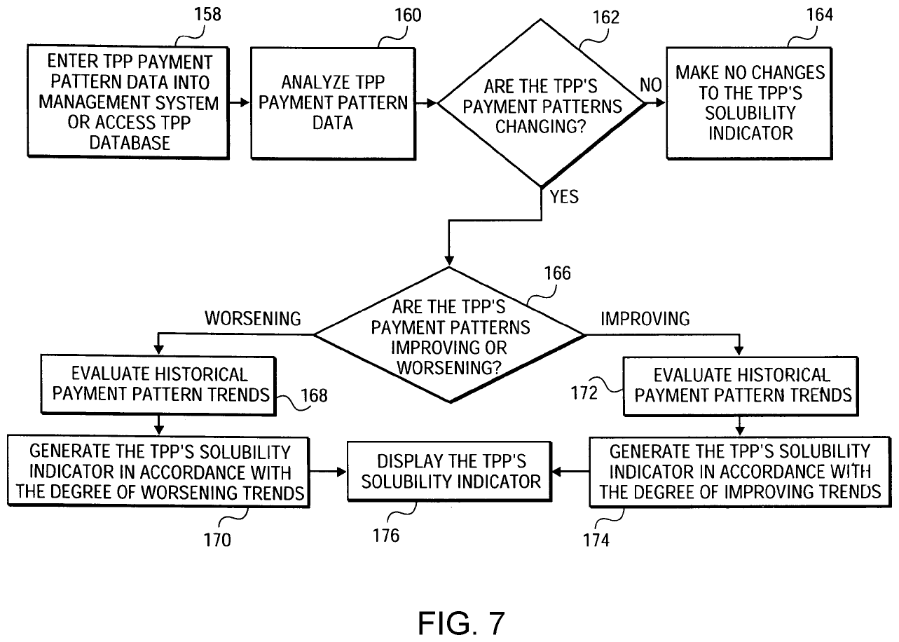

The evaluation software of the system may also provide an indication of the estimated financial strength or solvency of the TPP based on recent trends in the TPP's payment patterns. As illustrated by the flow diagram of FIG. 7, to evaluate the solvency of a TPP, a medical services provider staff member enters data relating to a TPP's payment patterns into a management system access terminal or otherwise accesses the management system's TPP database 158. The management system, or one of its associated access terminals operating with appropriate software, analyzes the TPP's payment pattern data 160 and determines whether the TPP's payment patterns are changing over time 162. If the TPP's payment patterns are not changing, the software generates a risk indicator for the TPP based upon its payment patterns or otherwise indicates 164 that there is no apparent indication of a threat of insolvency. If there are changes in the TPP's payment patterns, the software evaluates whether those changes are improving the payment patterns of the TPP, or whether the payment patterns are getting worse 166. If the payment patterns are worsening, the software evaluates the historical payment pattern trends 168, such as extreme recent changes in payment patterns, moderate changes in payment patterns over a long period of time, or regular periodic improving and worsening of payment patterns. The software then generates a risk indicator for the TPP in accordance with the degree of worsening payment pattern trends to represent the threat of the TPP becoming insolvent. Low risk rankings may indicate a likelihood of the TPP becoming insolvent soon, or that the TPP has difficulty paying its bills on time or in full, and high risk rankings may indicate a relatively smaller likelihood that the TPP will ever become insolvent, or that the TPP pays its bills on time and in full. The purpose behind using the historical payment patterns of a TPP to determine the likelihood of the TPP becoming insolvent is the trend of TPPs to begin adjusting their payment patterns to postpone their immediate expenditures in an attempt to remain solvent. Similarly, if the TPP's payment patterns are improving, the software evaluates the historical payment pattern trends 172, and generates a risk indicator for the TPP in accordance with the degree of the improving trends 174. Once a risk indicator has been generated, it is displayed to the staff member 176.

Once the patient has been accepted as a patient, the patient's information has already been entered into the system by a medical services provider staff member entering the patient's demographic information 104 into the computer terminal for association with the patient database. Alternatively, the patient may be provided with a wireless access terminal configured with software to display an electronic form which the patient may fill-out to include the patient's medical history, guarantor, and other necessary demographic information. The computer terminal or wireless access terminal of the present invention is in communication with the TPP and patient databases through an Internet connection so that all of the information in those databases may be available to authorized users at many locations.

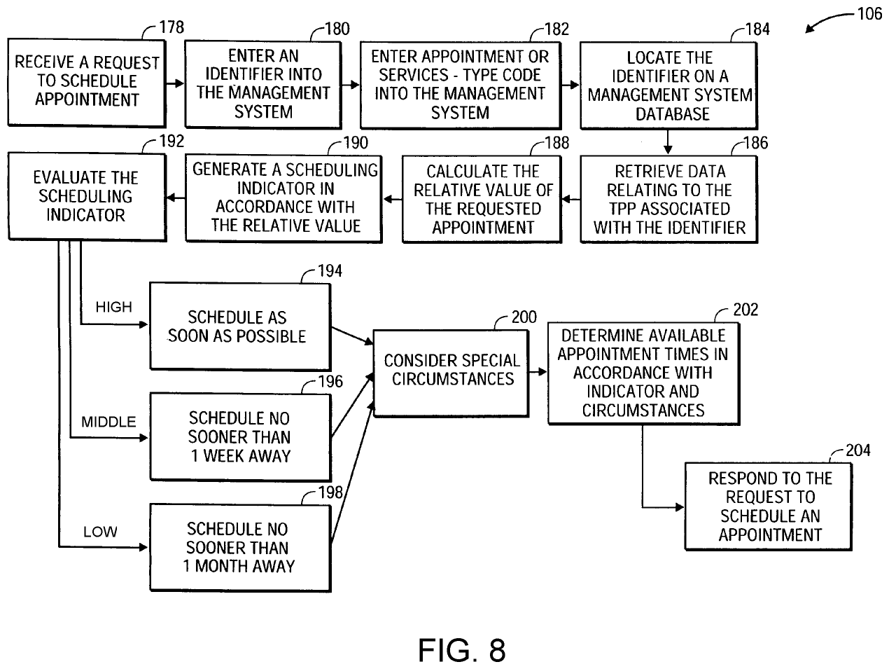

After a patient is accepted as a new patient and has the required demographic information stored in the patient database, at some point the patient will likely desire to schedule an appointment 106. As illustrated by the flow diagram in FIG. 8, when a patient calls in to schedule an appointment 178, a medical services provider staff member with access to an appropriately configured computer terminal will receive the call and enter a patient identifier 180 such as the patient's name and/or TPP plan number into the terminal to access the patient's information. The staff member may then also enter a predetermined code for the type of appointment or medical services the patient is requesting 182, and with which physician the patient would like to visit. Appropriately configured software operating on the access terminal searches a management system patient database to locate the patient identifier which was entered 184, and correspondingly retrieves a TPP identifier and associated data relating to the patient's TPP 186. Using at least the factors found in Table 1, the software calculates the relative value of the requested appointment 188 to the medical services provider, and generates an appropriate scheduling indicator 190 in accordance with that calculated relative value. The scheduling indicator is displayed on an access terminal display for the staff member to evaluate 192 prior to responding to the request to schedule the appointment 204.

According to the embodiment shown in FIG. 8, a high scheduling indicator represents an indication that the requested appointment will likely be very profitable for the medical services provider and that an appointment should be scheduled as soon as possible 194. The system may operate to suggest the soonest available appointment time. A middle scheduling indicator represents an indication that the requested appointment should be scheduled no sooner than a predetermined time away, such as, but not limited to one week away 196, and a low indicator represents an indication that the appointment should be scheduled no sooner than a predetermined time away, such as, but not limited to than one month away 198. By scheduling appointments for the most profitable medical services first, or those with the highest relative value to the medical services provider, medical services providers may more effectively maximize their profits. In addition to the scheduling indicators, there may be other special circumstances 200 which should be considered by a staff member in scheduling an appointment. Such special circumstances may include the urgency of the treatment needed, the identity of the patient, other conditions which may be placed upon the patient to increase the likelihood of profitability for the medical services provider, and the like. The staff member then determines available appointment times in accordance with the scheduling indicator and special circumstances 202, and responds to the request to schedule an appointment 204.

In establishing acceptable parameters for appointment scheduling, resource scheduling, relative value calculations and the like, it is contemplated that a medical services provider may select from a menu of options to define at least a portion of the boundaries and data for the various indicators. Such boundaries and data may include data related to, but not limited to, a break-even point, or, more specifically, the operating costs for each individual procedure, the costs for various supplies needed for each procedure, the overhead costs for the facility, lost investment returns rates, collection costs at various points in collection, and the like.

Additionally, as part of scheduling, the medical services provider staff member may adjust the length of the visit within recommended or selectable limits for a particular visit type so that the visit request may raise a green indicator rather than an orange indicator, or an orange indicator rather than a red indicator. By adjusting the length of the visit to make the length of the visit more closely match the expected relative value of the TPP payment, patients who would otherwise have been unprofitable, may be seen. When the appointment is scheduled, the required assistant medical personnel, supplies, rooms, etc. are each automatically scheduled and may optionally be scheduled based, in part, upon the appointment value indicator. Preferably, the scheduling software automatically checks for the availability of the staff, supplies, rooms, etc. while checking for the availability of the primary medical provider. The system may automatically check to determine that a medical provider's access for an appointment does not exceed certain or predetermined numbers during a scheduling period to prevent overloading the scheduling period with less profitably payors.

While it is understood that embodiments of the present invention are useful with regard to scheduling of appointments, embodiments are not limited to only scheduling. The system may operate to utilize any information and/or data that is, or will be, available in order to maximize/optimize the benefit to medical providers. Benefit may be measured in dollars, quality of care or any other measure or metric that may trackable. Further, some embodiments contemplate the sharing the information that has been processed freely or for a fee in order to provide additional benefits to third parties as needed or requested.

Sometime before the patient arrives for the patient's appointment, a medical services provider staff member checks for a patient's medical records 107 on the patient database, and if the records are not there, orders them from a referring medical services provider or other previous medical services provider, or generates new patient medical record forms for the patient. If the records were not presently in the patient database but are available, the records may be entered into the database, scanned into the database, or otherwise included in the patient database for future use by authorized personnel.

The system has the ability to provide the cost for services that the patient is responsible for prior to the services being provided. However, at times, the services may change based on examination by the doctor. In these situations, after the patient's appointment is over, the patient returns to a medical services provider staff member and, if the patient has not already paid a required amount for the services, pays the outstanding amount, schedules further appointments as necessary, receives any reading material indicated by the primary medical personnel, receives a printed prescription if required and not automatically ordered, receives any other receipts or referral letters or records as necessary, and leaves.

If the patient's TPP plan requires a copay, a computer terminal accessible by the medical services provider staff member indicates clearly to the staff member that a copay amount is required and requires either an indication from the staff member that the copay amount has been paid or why the copay amount was not paid and when it will be paid.

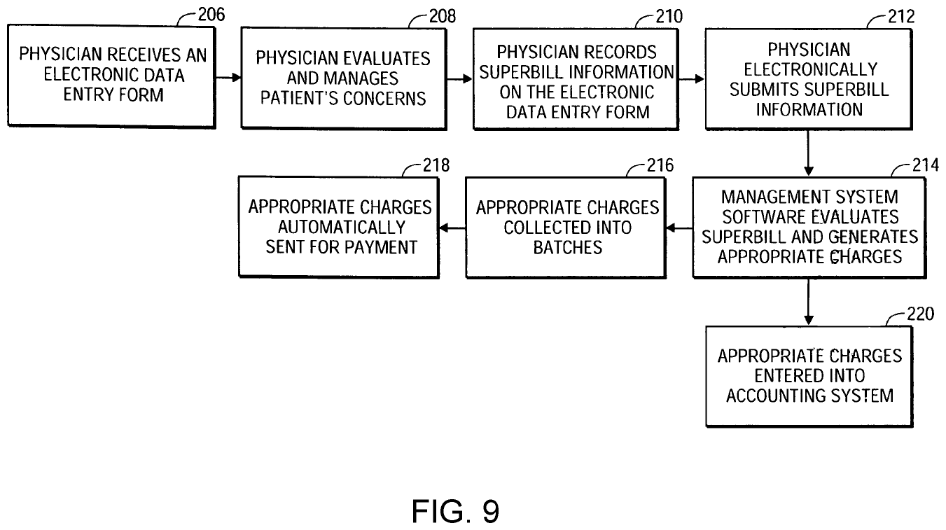

After the patient's appointment is completed, the billing process to collect the appropriate charges and fees for the visit is initiated 114. As illustrated by the flow diagram of FIG. 9, in an embodiment of the present invention, a primary medical personnel, such as a physician, is provided with an electronic data entry form 206. During a visit with a patient, the physician evaluates and manages patient concerns 208. Throughout the evaluation and management of the patient's concerns, the physician records the “superbill” information on the electronic data entry form 210 by indicating and/or selecting appropriate information as required by the form. Non-electronic “superbills” are commonly used and well known in the medical field to record patient-related charges for later billing.

When the electronic “superbill” form is complete and includes all appropriate charges, the physician, or other medical staff assistant, electronically submits the “superbill” information 212 for billing to the patient, TPP, or claims processing intermediary. The physician may submit the “superbill” by merely indicating that the appointment is complete, by pressing a button on the electronic data entry form, or by any other means known in the art for sending electronic data. The electronic data entry form may be displayed on a wireless access terminal 78, wireless interface 76 (FIG. 4), local or remote access terminal, or any other access terminal associated with a billing system. For the present embodiment, software operating in association with the management system evaluates the “superbill” information and generates the appropriate charges 214 for billing to the patient or TPP. Software operating within the central controller automatically enters the appropriate charges into the office management system's accounting software 220. For submitting the appropriate charges to the TPP or to a patient, the central controller either directly and immediately submits the “superbill” charges in appropriate form to the TPP over the Internet, modem, email or by other appropriate method, or bundles several “superbill” charges together in batch form 216 with other charges and data as required and automatically submit the information and charges to a TPP 218 at an appropriate time. Software which analyzes TPP reimbursement and treatment codes for medical services provided to select a code which optimizes reimbursement is well known in the art.

The present invention significantly improves payment billing time because a physician may enter the “superbill” for automatic billing without a data clerk to enter it into the system and send it out. A physician, during or immediately following an appointment, or even when performing a house call or other bed-side appointment where an accounting clerk may not be readily available, may record and submit an electronic “superbill” recorded on a portable device from anywhere access to the physician's accounting service may be obtained, including cellular transmission. By submitting the charges directly to the central controller for submission to the TPP at the next permissible interval rather than submitting a handwritten “superbill” to a data entry clerk for entry into a computer for submission to the TPP, the charges for a patient's visit more quickly reach the TPP for payment, and do not have the errors which may occur during the data entry process.

With the patient charges information appropriately recorded in the office management system's accounting software, the charges may be tracked by the system for fee collection purposes 116. The charges are compared with the allowable charges for the TPP, and the billing rules for the TPP are checked to determine if a bill should be provided directly to the patient with the patient's portion of the charges or if the TPP will directly pay the full bill.

As will be clear to one of ordinary skill in the art, the software of the various embodiments of the present invention will incorporate financial calculating, scheduling, evaluation, security, integration of a variety of systems, and other aspects of data analysis and comparison which may readily be programmed by software programmers of ordinary skill in their respective arts using ordinary algorithms and programming modules. The hardware and much of the software components required to establish a system configured as described herein are available generally and may be programmed and configured according to the various embodiments of the present invention by those of ordinary skill in the art. As will also be clear to one of ordinary skill in the art, the data analysis and other calculations required by the present invention may be done at the site of the medical services provider by providing appropriate software on-site and accessing the required data from an information service provider, or may alternatively be done at the site of the information service provider in response to a request by the medical services provider and an appropriately configured data stream or other report distributed back to the medical services provider, for example software on-site may provide access to a server, wherein the data and processing software is stored on the server and all operation of the software for utilizing the system occurs on the server and is displayed on a computer terminal at the service provider. Further, the computer terminal at a service provider may access the system through the internet or a web application and the operation of the software occurs at the server.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A value-based scheduling system comprising:
a server having a memory for storing and maintaining patient data and third party payors data; and
a computer terminal coupled to the server, wherein the computer terminal accesses memory of the server to store new patient data and to access stored third party payors data, wherein the computer terminal is programmed to:
initiate the value based scheduling system in response to a request to schedule an appointment for a patient and automatically establishing communication between the computer terminal and the server;
automatically generate a patient rank in response to automatically analyzing and evaluating the patient data and the third party payor data, wherein the patient data comprises a) a Cash Patient Grade; b) a Credit Card on File Grade; c) an Available Credit Grade; d) a Patient Credit Score Grade; e) a Health Savings Account/Medical Savings Account (HAS/MSA) Balance Grade; f) a Patient Amount Due Grade; g) a Prior Balance Due Grade; and h) a Pre-payment Amount Grade and the third party payor data comprises a) a third party payors' net present value grade; and b) a third party payors' weighting grade;
automatically accept the request to schedule the appointment in response to the patent rank being at greater than or equal to a predetermined value or automatically reject the request to schedule the appointment in response to the patient rank having a patient rank less than the predetermined value and display same at the computer terminal, wherein after accepting the request to schedule the appointment for the patient, the computer terminal is further programmed to generate a relative value of a medical service to be scheduled for the patient in response to analyzing and evaluating one or more factors comprising an average time for payment for the medical service; an average percentage of allowable billed charges paid for the medical service; an allowable fees schedule; a number of patients associated with the third party payor generally and within a particular region; a required copay amount for this or other patients; a total makeup of patient demographics for the services provider employing the system; and activity-based costs involved in providing the medical service for the patient of a specific third party payor and display same at the computer terminal; and
wherein in response to generating a patient rank and a relative value of services, automatically suggesting a day, week and or month for scheduling the patient based on the patient rank and relative value of the medical service to be provided; automatically suggesting the resources to use for the medical service to be provided and display same at the computer terminal; and automatically suggesting an amount of time for to schedule the patient based on the patient rank and the relative value of the medical service to be provided and display same at the computer terminal.

2. The system of claim 1, further comprising the computer terminal programmed to predict future insolvency of a third party payor in response to analyzing stored historical data of third party payors, wherein the historical data includes delay in payments made, partial payments made, percentage of allowable billed fees paid, denying payments, down-coding claims, increasing premiums, increasing co-pays, slowing authorizations for services, limiting the number of allowable visits, shifting specialty care to primary care physicians, linking third party payor enrollment with physician reimbursement, shifting financial risk to physicians, and forcing physicians to see patients after the third party payor stops paying and display same at the computer terminal.

3. A value-based scheduling system comprising:
a server having a memory for storing and maintaining patient data and third party payors data; and
a computer terminal coupled to the server, wherein the server is programmed to:
initiate the value based scheduling system in response to receiving a request to schedule an appointment for a patient from the computer terminal;
automatically generate a patient rank in response to automatically analyzing and evaluating the patient data and the third party payor data, wherein the patient data comprises a) a Cash Patient Grade; b) a Credit Card on File Grade; c) an Available Credit Grade; d) a Patient Credit Score Grade; e) a Health Savings Account/Medical Savings Account (HAS/MSA) Balance Grade; f) a Patient Amount Due Grade; g) a Prior Balance Due Grade; and h) a Pre-payment Amount Grade and the third party payor data comprises a) a third party payors' net present value grade; and b) a third party payors' weighting grade;
automatically accept the request to schedule the appointment in response to the patent rank being at greater than or equal to a predetermined value or automatically reject the request to schedule the appointment in response to the patient rank having a patient rank less than the predetermined value, wherein after accepting the request to schedule the appointment for the patient, the server is further programmed to generate a relative value of a medical service to be scheduled for the patient in response to analyzing and evaluating one or more factors comprising an average time for payment for the medical service; an average percentage of allowable billed charges paid for the medical service; an allowable fees schedule; a number of patients associated with the third party payor generally and within a particular region; a required copay amount for this or other patients; a total makeup of patient demographics for the services provider employing the system; and activity-based costs involved in providing the medical service for the patient of a specific third party payor and send the same for display on the computer terminal; and
wherein in response to generating a patient rank and a relative value of services, automatically suggesting a day, week and or month for scheduling the patient based on the patient rank and relative value of the medical service to be provided; automatically suggesting the resources to use for the medical service to be provided and display same at the computer terminal; and automatically suggesting an amount of time for to schedule the patient based on the patient rank and the relative value of the medical service to be provided and display same at the computer terminal.

4. The system of claim 3, further comprising the computer terminal programmed to predict future insolvency of a third party payor in response to analyzing stored historical data of third party payors, wherein the historical data includes delay in payments made, partial payments made, percentage of allowable billed fees paid, denying payments, down-coding claims, increasing premiums, increasing co-pays, slowing authorizations for services, limiting the number of allowable visits, shifting specialty care to primary care physicians, linking third party payor enrollment with physician reimbursement, shifting financial risk to physicians, and forcing physicians to see patients after the third party payor stops paying and send the same for display on the computer terminal.
